(19) United States
(12) Patent Application Publication
(10) Pub. No.: US 2022/0025152 A1
(43) Pub. Date: Jan. 27, 2022

(54) CROSSLINKED MATERIAL FOR ENDOSCOPE, ENDOSCOPE, AND COMPOSITION FOR FORMING CROSSLINKED MATERIAL FOR ENDOSCOPE

(71) Applicants: FUJIFILM Corporation, Tokyo (JP); Zeon Corporation, Tokyo (JP)

(72) Inventors: Yoshihiro NAKAI, Ashigarakami-gun (JP); Yuta KAWAMOTO, Ashigarakami-gun (JP); Yoshihisa TAKEYAMA, Tokyo (JP); Masahiro UENO, Tokyo (JP)

(73) Assignees: FUJIFILM Corporation, Tokyo, JP (unknown); Zeon Corporation, Tokyo (JP)

(21) Appl. No.: 17/496,254

(22) Filed: Oct. 7, 2021

Related U.S. Application Data
(63) Continuation of application No. PCT/JP2020/015577, filed on Apr. 6, 2020.

(30) Foreign Application Priority Data
Apr. 9, 2019 (JP) 2019-073996

(51) Int. Cl. C08K 3/04; A61L 29/12; C08J 3/24

(52) U.S. Cl. CPC C08K 3/041; A61L 29/126; C08J 3/24; C08K 2201/003; C08K 2201/011; C08K 2201/014; C08K 2201/006; A61L 2400/12

(57) ABSTRACT

A crosslinked material for an endoscope, containing a fluorinated elastomer and fibrous carbon nanostructures including single-walled carbon nanotubes, in which the amount of the fibrous carbon nanostructures in the crosslinked material is 0.1 parts by mass or more and less than 2.0 parts by mass per 100 parts by mass of the fluorinated elastomer, and a durometer type A hardness at 23° C. measured in accordance with JIS K 6253-3:2012 is 75A or less;
an endoscope using the crosslinked material for an endoscope; and
a composition for forming the crosslinked material for an endoscope.

### CROSS-REFERENCE TO RELATED APPLICATIONS

This application is a Continuation of PCT International Application No. PCT/JP2020/015577 filed on Apr. 6, 2020, which claims priority under 35 U.S.C. § 119 (a) to Japanese Patent Application No. 2019-073996 filed in Japan on Apr. 9, 2019. Each of the above applications is hereby expressly incorporated by reference, in its entirety, into the present application.

### FIELD OF THE INVENTION

The present invention relates to a crosslinked material for an endoscope, an endoscope, and a composition for forming a crosslinked material for an endoscope.

### BACKGROUND OF THE INVENTION

Endoscopes are medical devices for examining the inside of the body cavity, the inside of the digestive tract, the esophagus, or the like of a patient. Since endoscopes are inserted and used in the body, it is desirable to provide endoscopes that do not damage organs or cause pain or discomfort to a patient. In view of such a requirement, a spiral tube formed by winding a soft, bendable metal strip in a spiral form is adopted as a flexible tube that forms an insertion section (structural section to be inserted into a body) of an endoscope. Furthermore, the periphery of the spiral tube is covered with a flexible resin, and this resin cover layer is covered with a tube, so that the spiral tube does not cause stimulation or damage to an inner surface of, for example, the esophagus, digestive tract, or body cavity.

For example, Patent Literature 1 discloses a medical tube usable for an endoscope. It is disclosed that this tube is made of a medical composite material obtained by dispersing a carbon nanotube having an outer surface or an inner surface any of which is 30 nm or more and 200 nm or less in diameter in a polymeric material, the tube has low frictional property to living bodies, and the tube is less likely to be accumulated or remain in the living bodies even when the carbon nanotube comes off.

Requirements for durability and the like of flexible tubes for endoscopes have been enhanced year by year.

For example, a flexible tube for an endoscope is inserted into a body and repeatedly used in the inserted state while being, for example, bent or rotated. Therefore, a polymeric material for the tube or the like that forms the flexible tube for an endoscope is required to have both of a flexibility durable to bending with a large curvature and an appropriate repulsion force to return to the original straight shape, and have a property of being less likely to be broken even with the repeated bending.

The flexible tube for an endoscope is repeatedly disinfected using chemicals every time when used. Especially, in a case of being inserted to a site having a high infection possibility, such as a bronchus, the cleanliness at a sterilization level over disinfection is required. Accordingly, the polymeric material for the tube or the like that forms the flexible tube for an endoscope is required to have a high durability for enduring the sterilization treatment with hydrogen peroxide plasma or the like performed every time when used.

### CITATION LIST

### Patent Literatures

Patent Literature 1: JP-A-2009-39439 (“JP-A” means an unexamined published Japanese patent application)

### SUMMARY OF THE INVENTION

### Technical Problem

Patent Literature 1 discloses that the medical tube can be subjected to the bending with the curvature radius 30 mm. However, through examinations by the inventors, it has been found that the medical tube of Patent Literature 1 tends to be broken by the repeated bending operation when the medical tube is used as a constituting member of a flexible tube for an endoscope. In addition, the medical tube has not achieved the sufficient durability to the sterilization treatment.

The present invention provides a crosslinked material appropriate for a constituting material of an endoscopic tube (outer cover) that has a sufficient flexibility as a constituting member of an endoscope, has an excellent tear strength, is less likely to be broken regardless of repeated bending operation, and further, is excellent in sterilization resistance, an endoscope using the crosslinked material, and a composition appropriate for forming the crosslinked material.

### Solution to Problem

The inventors have been examined the endoscopic tube for ensuring the above-described properties, and found that the above-described problems can be solved by adding fibrous carbon nanostructures including single-walled carbon nanotubes to a fluorinated elastomer by a predetermined amount, and setting a hardness of a crosslinked material obtained by introducing crosslinked structures thereto to a value in a predetermined range. The inventors have further conducted studies on the basis of these findings and completed the present invention.

The objects of the present invention have been achieved by the following means.

<1>

A crosslinked material for an endoscope, containing:

a fluorinated elastomer; and

fibrous carbon nanostructures including single-walled carbon nanotubes,

wherein the amount of the fibrous carbon nanostructures in the crosslinked material is 0.1 parts by mass or more and less than 2.0 parts by mass per 100 parts by mass of the fluorinated elastomer, and
wherein a durometer type A hardness at 23° C. measured in accordance with JIS K 6253-3:2012 is 75A or less.
<2>

The crosslinked material for an endoscope described in the above item <1>, wherein the fibrous carbon nanostructures exhibit a convex upward shape in a t-plot obtained from an adsorption isotherm.

<3>

The crosslinked material for an endoscope described in the above item <2>, wherein the t-plot has a bending point in a range of 0.2 nm s t s 1.5 nm.

<4>

The crosslinked material for an endoscope described in the above item <2> or <3>, wherein total specific surface area S1 and internal specific surface area S2 of the fibrous carbon nanostructures, obtained from the t-plot, satisfy the condition 0.05≤S2/S1≤0.30.

<5>

The crosslinked material for an endoscope described in any one of the above items <1> to <4>, wherein the fibrous carbon nanostructures have an average diameter of 2 nm or more and 10 nm or less.

<6>

The crosslinked material for an endoscope described in any one of the above items <1> to <5>, wherein the amount of the fibrous carbon nanostructures in the crosslinked material is 0.1 parts by mass or more and less than 1.6 parts by mass per 100 parts by mass of the fluorinated elastomer.

<7>

The crosslinked material for an endoscope described in any one of the above items <1> to <6>, containing a carbon black,

wherein the amount of the carbon black in the crosslinked material is 5 parts by mass or more and 15 parts by mass or less per 100 parts by mass of the fluorinated elastomer.
<8>

An endoscope using the crosslinked material for an endoscope described in any one of the above items <1> to <7>.

<9>

A composition for forming the crosslinked material for an endoscope described in any one of the above items <1> to <7>, containing:

a fluorinated elastomer,

fibrous carbon nanostructures including single-walled carbon nanotubes; and

an organic peroxide.

### Advantageous Effects of Invention

The crosslinked material for an endoscope of the present invention has a sufficient flexibility as a constituting member of an endoscope, has an excellent tear strength, is less likely to be broken regardless of repeated bending operation, and further, is excellent in sterilization resistance.

The endoscope of the present invention includes the above crosslinked material for an endoscope, has a sufficient flexibility, has an excellent tear strength, is less likely to be broken regardless of repeated bending operation, and further, is excellent in sterilization resistance.

The composition for forming a crosslinked material for an endoscope of the present invention is appropriate for forming the crosslinked material for an endoscope.

### BRIEF DESCRIPTION OF THE DRAWING

FIG. 1 is an external view illustrating a configuration of an electronic endoscope according to an embodiment.

### DESCRIPTION OF EMBODIMENTS

### (Crosslinked Material for Endoscope)

The crosslinked material for an endoscope of the present invention includes a fluorinated elastomer and fibrous carbon nanostructures, and is a crosslinked material in which crosslinked structures are introduced to at least a part of the fluorinated elastomer.

In the crosslinked material for an endoscope of the present invention, the amount of the fibrous carbon nanostructures is 0.1 parts by mass or more and less than 2.0 parts by mass per 100 parts by mass of the fluorinated elastomer. A durometer type A hardness of the crosslinked material at 23° C. measured in accordance with JIS K 6253-3:2012 is 75A or less. The “amount of fluorinated elastomer” is a sum of an amount of uncrosslinked fluorinated elastomer and an amount of the fluorinated elastomer in which the crosslinked structures are formed when the uncrosslinked fluorinated elastomer is present.

It is considered that the crosslinked material for an endoscope of the present invention ensures the excellent tear strength and bending resistance while having the sufficient flexibility as the constituting material of an endoscope because of a network structure formed by the fibrous carbon nanostructures including the single-walled carbon nanotubes and the hardness of the crosslinked material in the specific range. Furthermore, it is considered that the fibrous carbon nanostructures have radical scavenging abilities, and thus effectively contributes to suppressing the decomposition of the fluorinated elastomer in the sterilization treatment or the like.

The following describes components included and components that may be included in the crosslinked material for an endoscope of the present invention.

### <Fluorinated Elastomer>

The fluorinated elastomer used for the crosslinked material for an endoscope of the present invention is not particularly limited, and one generally used as a constituting member of an endoscope can be widely used. Specific examples of the fluorinated elastomer include vinylidene fluoride-series rubber (FKM), tetrafluoroethylene-propyene-series rubber (FEPM), tetrafluoroethylene-perfluoromethyl vinyl ether-series rubber (FFKM), and tetrafluoroethylene-series rubber (TFE). These fluorinated elastomers can be used alone or in combination of two or more.

Among these, as the fluorinated elastomer, preferred are vinylidene fluoride-series rubber (FKM) and tetrafluoroethylene-propylene-series rubber (FEPM), with tetrafluoroethylene-propylene-series rubber (FEPM) being more preferred.

The vinylidene fluoride-series rubber (FKM) is a fluororubber which contains vinylidene fluoride as a main component and shows superior characteristics such as heat resistance, oil resistance, chemical resistance, solvent resistance, and workability. Examples of the FKM include, but not particularly limited to, copolymers of vinylidene fluoride and hexafluoropyrene, terpolymers of vinylidene fluoride, hexafluoropyrene and tetrafluoroethylene, and quaterpolymers of vinylidene fluoride, hexafluoropyrene, tetrafluoroethylene and a vulcanization site monomer. Commercially available products of the FKM include “Viton” (Viton is a registered trademark in Japan, other countries, or both) manufactured by Chemours, and “DAI-EL G” (DAI-EL is a registered trademark in Japan, other countries, or both) manufactured by Daikin Industries, Ltd.

Preferred are quaterpolymers of vinylidene fluoride, hexafluoropyrene, tetrafluoroethylene and a vulcanization site monomer. The quaterpolymers are commercially available under the trade name “Viton GBL-200S” manufactured by Chemours, for example.

The tetrafluoroethylene-propylene-series rubber (FEPM) is a fluororubber which is based on an alternating copolymer of tetrafluoroethylene (TFE) and propylene (P) and exhibits superior characteristics such as heat resistance, chemical resistance, polar solvent resistance, and steam resistance. Examples of the FEPM include, but not particularly limited to, copolymers of tetrafluoroethylene (TFE) and propylene (P), terpolymers of tetrafluoroethylene (TFE), propylene (P) and vinylidene fluoride (VdF), and terpolymers of tetrafluoroethylene (TFE), propylene (P) and a cure site monomer (CSM). Commercially available products of the copolymers of tetrafluoroethylene (TFE) and propylene (P) include “AFLAS 100” (AFLAS is a registered trademark in Japan, other countries, or both) and “AFLAS 150” manufactured by AGC Inc. Commercially available products of the terpolymers of tetrafluoroethylene (TFE), propylene (P) and vinylidene fluoride (VdF) include “AFLAS 200” manufactured by AGC Inc. Commercially available products of the terpolymers of tetrafluoroethylene (TFE), propylene (P) and a cure site monomer (CSM) include “AFLAS 300” manufactured by AGC Inc.

### <Fibrous Carbon Nanostructures>

Examples of the fibrous carbon nanostructures include cylindrical carbon nanostructures such as carbon nanotubes (CNTs) and non-cylindrical carbon nanostructures such as those formed of a network of 6-membered carbon rings in a flattened cylindrical shape. In the crosslinked material for an endoscope of the present invention, fibrous carbon nanostructures including single-walled CNTs are used.

The amount of the fibrous carbon nanostructures in the crosslinked material for an endoscope of the present invention needs to be 0.1 parts by mass or more, preferably 0.2 parts by mass or more, more preferably 0.3 parts by mass or more, and further preferably 0.4 parts by mass or more, per 100 parts by mass of the fluorinated elastomer. With the crosslinked material for an endoscope of the present invention including 0.1 parts by mass or more of the fibrous carbon nanostructures per 100 parts by mass of the fluorinated elastomer, the sufficient tear strength can be ensured.

Further, the amount of the fibrous carbon nanostructures in the crosslinked material for an endoscope of the present invention needs to be less than 2 parts by mass, preferably less than 1.8 parts by mass, more preferably less than 1.6 parts by mass, and further preferably 1.5 parts by mass or less, per 100 parts by mass of the fluorinated elastomer. By setting the upper limit value of the amount to less than 2 parts by mass, the crosslinked material for an endoscope of the present invention can be provided with the sufficient flexibility.

The fibrous carbon nanostructures including single-walled CNTs used in the crosslinked material for an endoscope of the present invention can be any fibrous carbon nanostructures so long as single-walled CNTs are included. The fibrous carbon nanostructures including single-walled CNTs may be those consisting only of single-walled CNTs, a mixture of single-walled CNTs and multi-walled CNTs, or a mixture of CNTs including at least single-walled CNTs and fibrous carbon nanostructures other than CNTs.

From an aspect of improving the flexibility and the tear strength of the crosslinked material for an endoscope of the present invention and an aspect of making the crosslinked material for an endoscope of the present invention more less likely to be broken regardless of the repeated bending operation, the number of single-walled CNTs in 100 fibrous carbon nanostructures is preferably 50 or more, more preferably 70 or more, and further preferably 90 or more.

It is also preferred that the fibrous carbon nanostructures including single-walled CNTs exhibit a convex upward shape in a t-plot obtained from an adsorption isotherm. The use of fibrous carbon nanostructures exhibiting a convex upward shape in the t-plot obtained from an adsorption isotherm makes it possible to form a shaped article having further increased flexibility.

It is preferred that the fibrous carbon nanostructures including single-walled CNTs have not undergone CNT opening treatment and exhibit a convex upward shape in a t-plot.

Adsorption generally refers to a phenomenon in which gas molecules are taken away from the gas phase to a solid surface, and is classified as physical or chemical adsorption depending on the cause of adsorption. The nitrogen gas adsorption method used to acquire a t-plot utilizes physical adsorption. In general, when adsorption temperature is constant, the number of nitrogen gas molecules adsorbed to the fibrous carbon nanostructures increases with increasing pressure. A plot of the adsorbed amount of nitrogen versus relative pressure (ratio of pressure P at adsorption equilibrium to saturated vapor pressure PO) refers to an “isotherm.” An isotherm obtained when the adsorbed amount of nitrogen gas is measured while increasing pressure refers to an “adsorption isotherm” and an isotherm obtained when the adsorbed amount of nitrogen gas is measured while decreasing pressure refers to a “desorption isotherm.”

The t-plot is obtained by converting relative pressure to average adsorbed nitrogen gas layer thickness t (nm) in an adsorption isotherm measured by the nitrogen gas adsorption method. Specifically, an average adsorbed nitrogen gas layer thickness t corresponding to a given relative pressure is calculated from a known standard isotherm of average adsorbed nitrogen gas layer thickness t plotted against relative pressure P/PO and the relative pressure is converted to the corresponding average adsorbed nitrogen gas layer thickness t to obtain a t-plot for the fibrous carbon nanostructures (t-plot method of de Boer et al.).

The growth of an adsorbed layer of nitrogen gas for a sample having pores at the surface is divided into the following processes (1) to (3). The gradient of the t-plot changes according to the following processes (1) to (3):

(1) a process in which a single molecular adsorption layer is formed over the entire surface by nitrogen molecules;
(2) a process in which a multi-molecular adsorption layer is formed in accompaniment to capillary condensation filling of pores; and
(3) a process in which a multi-molecular adsorption layer is formed on a surface that appears to be non-porous due to the pores being filled by nitrogen.

It is preferred that the t-plot for the fibrous carbon nanostructures including single-walled CNTs shows a straight line crossing the origin in a region in which the average adsorbed nitrogen gas layer thickness t is small and deviates downward from the straight line as t increases to have a convex upward shape. Such a t-plot shape indicates that the ratio of internal specific surface area to total specific surface area of the fibrous carbon nanostructures is large, indicating the presence of a large number of openings formed in the carbon nanostructures that constitute the fibrous carbon nanostructures. As a result, it is presumed that this further increases the flexibility of the crosslinked material for an endoscope of the present invention formed using such fibrous carbon nanostructures.

It is preferred that the t-plot for the fibrous carbon nanostructures including single-walled CNTs has a bending point in a range of 0.2 nm s t s 1.5 nm, more preferably in a range of 0.45 nm s t s 1.5 nm, and further preferably in a range of 0.55 nm s t s 1.0 nm. When the position of the bending point of the t-plot falls within the range described above, it is possible to further increase tear strength and sterilization resistance as the characteristics of the fibrous carbon nanostructures further improve.

The “position of the bending point” is an intersection point of an approximate straight line A for the process (1) and an approximate straight line B for the process (3) in the t-plot.

The fibrous carbon nanostructures including single-walled CNTs have a ratio of internal specific surface area S2 to total specific surface area S1 (S2/S1), obtained from the t-plot, of preferably 0.05 or more, more preferably 0.06 or more, and further preferably 0.08 or more, but preferably 0.30 or less. When the value of S2/S1 is 0.05 or more and 0.30 or less, it is possible to further increase tear strength and sterilization resistance as the characteristics of the fibrous carbon nanostructures further improve.

The fibrous carbon nanostructures including single-walled CNTs can have any total specific surface area S1 and any internal specific surface area S2. However, S1 is preferably 600 m²/g or more and 1,400 m²/g or less, and more preferably 800 m²/g or more and 1,200 m²/g or less. On the other hand, S2 is preferably 30 m²/g or more and 540 m²/g or less.

Total specific surface area S1 and internal specific surface area S2 of the fibrous carbon nanostructures including single-walled CNTs can be found from the t-plot. Specifically, first, total specific surface area S1 can be found from the gradient of an approximate straight line corresponding to the process (1) and external specific surface area S3 can be found from the gradient of an approximate straight line corresponding to the process (3). Internal specific surface area S2 can then be calculated by subtracting external specific surface area S3 from total specific surface area S1.

Measurement of adsorption isotherm, preparation of the t-plot, and calculation of the total specific surface area S1 and the internal specific surface area S2 based on t-plot analysis for the fibrous carbon nanostructures including single-walled CNTs can be made using, for example, BELSORP-mini (BELSORP is a registered trademark in Japan, other countries, or both), a commercially available measurement instrument available from Bel Japan Inc.

The fibrous carbon nanostructures including single-walled CNTs are preferably those having a ratio of a standard deviation (a) of diameters multiplied by 3 (3σ) to average diameter (Av) (3σ/Av) of greater than 0.20 and less than 0.60, more preferably those having 3σ/Av of greater than 0.25, and further preferably those having 3σ/Av of greater than 0.40. The use of fibrous carbon nanostructures including single-walled CNTs having 3σ/Av of greater than 0.20 and less than 0.60 makes it possible to form a shaped article which exhibits both further increased flexibility and tear strength.

“Average diameter (Av) of fibrous carbon nanostructures” and “standard deviation (σ) (σ: sample standard deviation) of diameters of fibrous carbon nanostructures” can each be obtained by measuring the diameters (outer diameters) of 100 fibrous carbon nanostructures randomly selected by transmission electron microscopy. The average diameter (Av) and standard deviation (σ) of the fibrous carbon nanostructures including single-walled CNTs may be adjusted either by changing the production method and/or the production conditions of the fibrous carbon nanostructures or by combining different types of fibrous carbon nanostructures prepared by different production methods.

In a Raman spectrum of the fibrous carbon nanostructures including single-walled CNTs, the ratio of G band peak intensity to D band peak intensity (G/D ratio) is preferably 1 or more and 20 or less. When the G/D ratio is 1 or more and 20 or less, it is possible to form a shaped article which exhibits both further increased flexibility and tear strength.

A lower limit of an average diameter (Av) of the fibrous carbon nanostructures including single-walled CNTs is preferably 0.8 nm or more, more preferably 2 nm or more, and further preferably 2.5 nm or more. An upper limit of Av is preferably 10 nm or less, more preferably 6 nm or less. When the average diameter (Av) of the fibrous carbon nanostructures is 2 nm or more, it is possible to form a shaped article which exhibits further increased tear strength. When the average diameter (Av) of the fibrous carbon nanostructures is 10 nm or less, it is possible to form a shaped article having further increased flexibility.

The fibrous carbon nanostructures including single-walled CNTs preferably have an average length at the time of synthesis of 100 μm or more. To reduce the damage, such as breakage and cutting, generated on the fibrous carbon nanostructures during the dispersion, an average length of the structures during the synthesis is preferably 5000 μm or less.

The fibrous carbon nanostructures including single-walled CNTs preferably have an aspect ratio (length/diameter) of greater than 10. The aspect ratio of the fibrous carbon nanostructures can be found by measuring diameters and lengths of 100 fibrous carbon nanostructures randomly selected by transmission electron microscopy and calculating the average of ratios of length to diameter (length/diameter).

The fibrous carbon nanostructures including single-walled CNTs preferably have a BET specific surface area of 600 m²/g or more, more preferably 800 m²/g or more, but preferably 2,500 m²/g or less, more preferably 1,200 m²/g or less. When the BET specific surface area of the fibrous carbon nanostructures including single-walled CNTs is 600 m²/g or more, it is possible to further increase tear strength as the strength of the formed shaped article can be increased. When the BET specific surface area of the fibrous carbon nanostructures including single-walled CNTs is 2,500 m²/g or less, it is possible to allow the formed shaped article to have a suitable hardness while maintaining its flexibility.

The term “BET specific surface area” as used herein refers to a nitrogen adsorption specific surface area measured by the BET method.

In accordance with the super growth method described later, the fibrous carbon nanostructures including single-walled CNTs are obtained, on a substrate having thereon a catalyst layer for carbon nanotube growth, in the form of an aggregate wherein fibrous carbon nanostructures are aligned substantially perpendicularly to the substrate (aligned aggregate). The mass density of the fibrous carbon nanostructures in the form of such an aggregate is preferably 0.002 g/cm³ or more and 0.2 g/cm³ or less. A mass density of 0.2 g/cm³ or less allows the fibrous carbon nanostructures to be homogeneously dispersed within the fluorinated elastomer because binding among the fibrous carbon nanostructures is weakened. A mass density of 0.002 g/cm³ or more improves the unity of the fibrous carbon nanostructures thus preventing the fibrous carbon nanostructures from becoming unbound and making the fibrous carbon nanostructures easier to be handled.

The fibrous carbon nanostructures including single-walled CNTs preferably include micropores. Preferred fibrous carbon nanostructures are those having micropores with a pore diameter of smaller than 2 nm and the abundance thereof as measured in terms of micropore volume determined by the method described below is preferably 0.40 mL/g or more, more preferably 0.43 mL/g or more, and further preferably 0.45 mL/g or more, with the upper limit being generally on the order of about 0.65 mL/g. The presence of such micropores in the fibrous carbon nanostructures including single-walled CNTs can further increase flexibility. Micropore volume can be adjusted, for example, by appropriately changing the preparation method and preparation conditions of the fibrous carbon nanostructures.

“Micropore volume (Vp)” can be calculated using Equation (I): Vp=(V/22414)×(M/ρ) by measuring a nitrogen adsorption isotherm of the fibrous carbon nanostructures including single-walled CNTs at liquid nitrogen temperature (77 K) with the amount of adsorbed nitrogen at a relative pressure P/P0 of 0.19 defined as V, where P is a measured pressure at adsorption equilibrium, and P0 is a saturated vapor pressure of liquid nitrogen at time of measurement. In Equation (I), M is a molecular weight of 28.010 of the adsorbate (nitrogen), and ρ is a density of 0.808 g/cm³ of the adsorbate (nitrogen) at 77 K. Micropore volume can be measured for example using “BELSORP-mini (trademark)” produced by Bel Japan Inc.

The fibrous carbon nanostructures including single-walled CNTs having the properties described above can be efficiently produced, for example, by forming a catalyst layer on a substrate surface by wet process in the super growth method (see WO 2006/011655) wherein during synthesis of CNTs through chemical vapor deposition (CVD) by supplying a feedstock compound and a carrier gas onto a substrate having thereon a catalyst layer for carbon nanotube production, the catalytic activity of the catalyst layer is dramatically improved by providing a trace amount of an oxidizing agent (catalyst activating material) in the system. Hereinafter, carbon nanotubes obtained by the super growth method may also be referred to as “SGCNTs.”

### <Carbon Black>

To more improve at least the tear strength and the sterilization resistance, the crosslinked material for an endoscope of the present invention preferably includes a carbon black. In the crosslinked material for an endoscope of the present invention, the amount of the carbon black is preferably 1 part by mass or more and 25 parts by mass or less, more preferably 3 parts by mass or more and 20 parts by mass or less, and 5 parts by mass or more and 15 parts by mass or less, per 100 parts by mass of the fluorinated elastomer.

### <Other Components>

The crosslinked material for an endoscope of the present invention may include components such as an additive within a range not impairing the effect of the present invention. Such a component is not particularly limited, and the additive such as a reinforcing material, a lubricant, an anti-aging agent, and a coupling agent are included. A compound derived from a cross-linking agent, a cross-linking aid, and a co-cross-linking agent that can be used in preparing the crosslinked material for an endoscope of the present invention may be included.

The reinforcing material is not particularly limited, and silica can be used, for example.

The lubricant is not particularly limited, and sodium stearate can be used, for example.

The anti-aging agent is not particularly limited, and examples thereof include 2,6-di-tert-butyl-p-cresol, pentaerythrityl-tetrakis[3-(3,5-di-t-butyl-4-hydroxyphenyl)propionate], 2,2′-methylenebis(4-methyl-6-t-butylphenyl), bis(2,2,6,6-tetramethyl-4-piperidyl)sebacate, and N,N′-(hexane-1,6-diyl)bis[3-(3,5-di-tert-butyl-4-hydroxyphenyl)propanamide].

The coupling agent is not particularly limited, and examples thereof include γ-chloropropyltrimethoxysilane, vinyltriethoxysilane, vinyl-tris-(β-methoxyethoxy)silane, γ-methacryloxypropyltrimethoxysilane, β-(3,4-ethoxycyclohexyl)ethyltrimethoxysilane, γ-glycidoxypropyttrimethoxysilane, γ-mercaptopropyltrimethoxysilane, γ-aminopropyltrimethoxysilane, and N-β-(aminoethyl)-γ-aminopropyltrimethoxysilane.

The cross-linking agent is not particularly limited, and cross-linking agents known in the art can be used which are able to crosslink the fluorinated elastomer contained in the crosslinked material for an endoscope of the present invention. Examples of such a cross-linking agent include peroxide-based cross-linking agents (organic peroxide), polyol-based cross-linking agents, and polyamine-based cross-linking agents.

The organic peroxide includes an ordinarily used organic peroxide that has at least an O—O bond and a carbon atom in a molecule, for example, hydroperoxide, dialkyl peroxide, peroxyester, diacyl peroxide, and peroxyketal.

Specific examples thereof include the following organic peroxides. Hydroperoxide: such as p-menthane hydroperoxide, diisopropylbenzene hydroperoxide, 1,1,3,3-tetramethylbutyl hydroperoxide, cumene hydroperoxide and t-butyl hydroperoxide.

Dialkyl peroxide: such as 1,3-bis(2-t-butylperoxyisopropyl)benzene, dicumyl peroxide, 2,5-dimethyl-2,5-di(t-butylperoxy)hexane, t-butyl cumyl peroxide, di-t-hexyl peroxide, di-t-butyl peroxide, and 2,5-bis(t-butylperoxy)-2,5-dimethyl-3-hexyne.

Peroxyester: such as t-butyl peroxybenzoate, t-butyl peroxymaleate, t-butyl peroxy-3,5,5-trimethylhexanoate, t-butyl peroxylaurate, t-butyl peroxyisopropyl monocarbonate, t-butyl peroxy-2-ethythexyl monocarbonate, t-hexyl peroxybenzoate, 2,5-dimethyl-2,5-di(benzoylperoxy)hexane, and t-butyl peroxyacetate.

Diacyl peroxide: such as bis(3-methyl benzoyl)peroxide, benzoyl(3-methyl benzoyl)peroxide, dibenzoyl peroxide, and bis(4-methylbenzoyl)peroxide.

Peroxyketal: such as 1,1-bis(t-hexylperoxy)-3,3,5-trimethylcyclohexane, 1,1-bis(t-hexylperoxy)cyclohexane, 1,1-bis(t-butylperoxy)-2-methylcyclohexane, 1,1-bis(t-butylperoxy)cyclohexane, 2,2-bis(t-butylperoxy)butane, n-butyl 4,4-bis(t-butylperoxy)valerate, and 2,2-bis(4,4-bis(t-butylperoxy)cycohexyl)propane.

The cross-linking aid is not particularly limited, and zinc oxide can be used, for example.

The co-cross-linking agent is not particularly limited, and triallyl isocyanurate can be used, for example.

One kind of the above-described other components may be used alone, or two kinds or more may be used in combination. The amount of the above-described other components in the crosslinked material for an endoscope of the present invention is not particularly limited insofar as the effect of the present invention is not impaired, and for example, 20 parts by mass or less, preferably 15 parts by mass or less is preferred, and more preferably 10 parts by mass or less, of the other components can be included per 100 parts by mass of the fluorinated elastomer.

### <Durometer Type A Hardness>

In the crosslinked material for an endoscope of the present invention, from an aspect of the flexibility of the endoscope that includes the crosslinked material, a durometer type A hardness of the crosslinked material at 23° C. measured in accordance with JIS K 6253-3:2012 is 75A or less, preferably 40A to 75A, more preferably 50A to 75A, and further preferably 60A to 75A.

The durometer type A hardness can have a value in a specific range by, for example, the type and the amount of the components included in the crosslinked material for an endoscope and the cross-linking density of the fluorinated elastomer.

### (Endoscope)

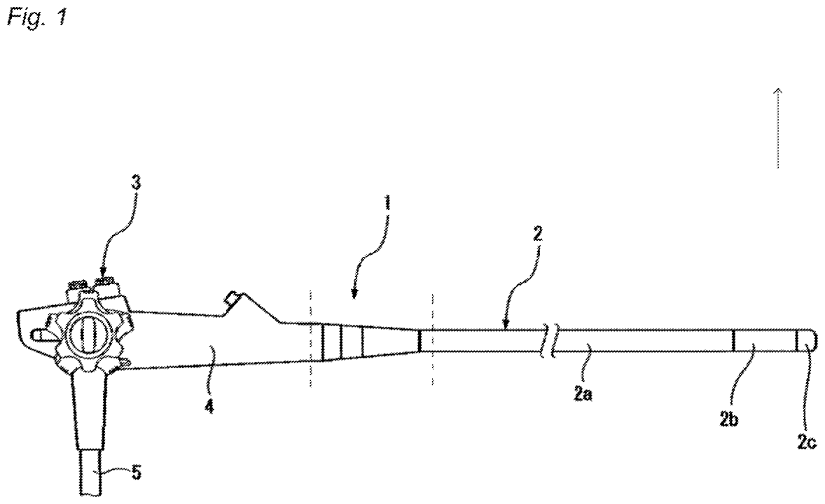

An electronic endoscope (endoscope) will now be described as an example of an endoscopic medical device using a crosslinked material for an endoscope according to a preferred embodiment of the present invention. An electronic endoscope includes a flexible tube for an endoscope, the flexible tube being incorporated in the electronic endoscope, and is used as a medical device for, for example, examining the inside of the body by inserting the flexible tube into the body. In the example illustrated in FIG. 1, an electronic endoscope 1 includes an insertion section 2 to be inserted into a body, a main body operating section 4 that is connected to a proximal end portion of the insertion section 2, and a universal cord 5 to be connected to a processor device or a light source device. The main body operating section 4 includes an air/water supply button 3. The insertion section 2 includes a flexible tube 2a connected to the main body operating section 4, an angle portion 2b connected to the flexible tube 2a, and a tip portion 2c which is connected to the distal end of the angle portion 2b and in which an imaging device (not shown) for imaging the inside of the body is installed. The flexible tube 2a that accounts for a large portion of the length of the insertion section 2 has flexibility across substantially the entire length thereof and is configured so that, in particular, a portion to be inserted into the inside of a body cavity or the like has higher flexibility.

The crosslinked material for an endoscope used for an endoscope of the present invention can be used in a shape appropriately adjusted corresponding to the shape of a position at which the crosslinked material for an endoscope is applied. For example, the crosslinked material for an endoscope of the present invention can be formed as a tube and be applied to the insertion section 2 as an angle rubber or a bend preventing rubber. The crosslinked material for an endoscope of the present invention may be formed as an O-ring and applied to the air/water supply button 3. The crosslinked material for an endoscope of the present invention is less likely to be broken even when attaching to and removal from the air/water supply button 3 are repeated.

### (Composition for Forming Crosslinked Material for Endoscope)

The composition for forming a crosslinked material for an endoscope of the present invention includes the fluorinated elastomer, the fibrous carbon nanostructures including the single-walled carbon nanotubes, and the organic peroxide, and the composition can be appropriately used for forming the crosslinked material for an endoscope of the present invention. The composition for forming a crosslinked material for an endoscope of the present invention may include the reinforcing material, the lubricant, the anti-aging agent, the coupling agent, the cross-linking aid, and the co-cross-linking agent described above.

The amounts of the components included in the composition for forming a crosslinked material for an endoscope of the present invention can be appropriately adjusted such that they become the amounts of the components included in the crosslinked material for an endoscope of the present invention.

### (Preparation Method of Crosslinked Material for Endoscope and Composition for Forming Crosslinked Material for Endoscope)

While the respective preparation methods of the crosslinked material for an endoscope of the present invention and the composition for forming a crosslinked material for an endoscope of the present invention are not particularly limited, the crosslinked material for an endoscope of the present invention is preferably formed through a cross-linking reaction caused in the composition for forming a crosslinked material for an endoscope of the present invention. The following describes the preferred preparation method of the composition for forming a crosslinked material for an endoscope of the present invention and the preferred preparation method of the crosslinked material for an endoscope of the present invention in this order.

### <Preparation of Composition for Forming Crosslinked Material for Endoscope>

The composition for forming a crosslinked material for an endoscope of the present invention can be prepared, for example, by mixing or kneading the fluorinated elastomer, the fibrous carbon nanostructures including single-walled carbon nanotubes, the organic peroxide, and any component described as the “Other components” as necessary with a desired combination percentage.

Specifically, the composition for forming a crosslinked material for an endoscope of the present invention can be prepared by any method by obtaining a mixture of the fluorinated elastomer and the fibrous carbon nanostructures including single-walled carbon nanotubes, and then kneading the resulting mixture at 20° C. to 100° C. with an organic peroxide and an optional component.

Preparation of the mixture of the fluorinated elastomer and the fibrous carbon nanostructures including single-walled carbon nanotubes can be effected by any mixing method capable of dispersing in the fluorinated elastomer the fibrous carbon nanostructures including single-walled carbon nanotubes. Specifically, the mixture can be prepared by any method by adding the fibrous carbon nanostructures including single-walled CNTs in a fluorinated elastomer solution which is obtained by dissolving the fluorinated elastomer into an organic solvent or in a fluorinated elastomer dispersion which is obtained by dispersing the fluorinated elastomer into a dispersion medium; dispersing the fibrous carbon nanostructures including the single-walled CNTs at 10° C. to 50° C.; and removing the organic solvent or dispersion medium from the resulting dispersed liquid.

The dispersing treatment can be carried out by dispersing methods known in the art. Examples of such a dispersing treatment include, but not particularly limited to, ultrasonic homogenizers, wet jet mills, and high-speed rotary shearing dispersers, with wet jet mills being preferred because a moderately strong shearing force can be applied to sufficiently disperse the fibrous carbon nanostructures to form a crosslinked material for an endoscope with improved material homogeneity. The pressure applied during the dispersing treatment of the mixture by wet jet mill is preferably 10 to 180 MPa, more preferably 15 to 170 MPa, more preferably 20 to 160 MPa, and further preferably 20 to 150 MPa. The number of dispersing treatments (number of passes) is 1 or more, preferably 2 to 20. The dispersion treatment temperature is preferably 0° C. to 80° C. Examples of the wet jet mills usable for the dispersing treatment include “NanoVater” (NanoVater is a registered trademark in Japan, other countries, or both) and “L-ES007 (trademark)” (each manufactured by Yoshida Kikai Co., Ltd.), “BERYU SYSTEM PRO” (manufactured by Beryu Corporation), ultrahigh-pressure wet atomizer (Yoshida Works Pro), “Nanomizer” (Nanomizer is a registered trademark in Japan, other countries, or both) (manufactured by Nanomizer, Inc.), and “StarBurst” (StarBurst is a registered trademark in Japan, other countries, or both) (manufactured by Sugino Machine Ltd.). From the viewpoint of limiting clogging, the minimum flow path diameter of the wet jet mill is preferably 100 μm or more, and from the viewpoint of achieving effective dispersing under pressure, the minimum flow path diameter is preferably 1,000 μm or less.

The mixture can be prepared by removing the organic solvent or dispersion medium from the resulting dispersed liquid. Removal of the organic solvent or dispersion medium can be carried by coagulation, casting or drying.

Kneading of the mixture with the organic peroxide and the arbitrary components can be carried out for example using a mixer, a single screw kneader, a twin screw kneader, a roll, Brabender (Brabender is a registered trademark in Japan, other countries, or both), or an extruder.

In thus obtained composition, the cross-linking reaction is substantially not caused because of preservation at low temperature (for example, −20° C.), thus allowing the stable preservation.

### <Preparation of Crosslinked Material for Endoscope>

The crosslinked material for an endoscope of the present invention can be obtained by shaping the composition for forming a crosslinked material for an endoscope into a desired form. Specifically, the crosslinked material for an endoscope of the present invention can be formed for example by placing the composition for forming a crosslinked material for an endoscope into a mold and cross-linking the composition.

### EXAMPLES

Hereafter, the present invention will be described in more detail by way of Examples. However, it is to be understood that the present invention is not limited to these Examples.

### <Preparation of Fibrous Carbon Nanostructures (B-1)>

The fibrous carbon nanostructures (B-1) used in Examples and Comparative Examples was prepared as follows.

In accordance with the descriptions of WO 2006/011655, the super growth method was used to prepare carbon nanotubes (SGCNTs) as the fibrous carbon nanostructures (B-1). Upon preparation of SGCNTs, formation of a catalyst layer on a substrate surface was carried out by the wet process and a source gas containing acetylene as a main component was used.

The obtained SGCNTs consisted primarily of single-walled CNTs, with the radial breathing mode (RBM) being observed in a low wavenumber range of 100 to 300 cm⁻¹ in a spectrum measured by a Raman spectrophotometer, which is characteristic of single-walled CNTs. The BET specific surface area of the SGCNTs as measured using a BET specific surface area meter (“BELSORP-max” (trademark) manufactured by Bel Japan Inc.) was 1,050 m²/g (unopened). The diameters and lengths of 100 SGCNTs randomly selected using a transmission electron microscope were measured to find the average diameter (Av), the standard deviation (σ) of the diameters and the average length for the SGCNTs. The average diameter (Av) was 3.3 nm, the standard deviation (σ) multiplied by 3 (3σ) was 1.9 nm, the ratio of 3σ to Av (3σ/Av) was 0.58, and the average length was 500 μm. A t-plot of the SGCNTs measured using “BELSORP-mini” (trademark) manufactured by Bel Japan Inc. was bent having a convex upward shape. The value of S2/S1 was 0.09 and the position t of the bending point was 0.6 nm.

### <Preparation of Composition for Forming Crosslinked Material for Endoscope>

### [Preparation of Composition for Forming Crosslinked Material for Endoscope According to Example 1]

The composition for forming a crosslinked material for an endoscope according to Example 1 shown in Table 1-1 below was prepared as follows.

### [Preparation of Mixture]

200 g of a fluorinated elastomer (A-1) was added to 4,000 g of methyl ethyl ketone as an organic solvent and stirred for 12 hours at room temperature to dissolve the fluorinated elastomer. Next, 0.2 g of fibrous carbon nanostructures (B-1) was added to the obtained fluorinated elastomer solution and the mixture was stirred for 15 minutes at room temperature using a stirrer (LABOLUTION (LABOLUTION is a registered trademark in Japan, other countries, or both) manufactured by PRIMIX Corporation). Further, using a wet jet mill (L-ES007 (trademark), manufactured by Yoshida Kikai Co., Ltd.), the solution containing the fluorinated elastomer (A-1) and the fibrous carbon nanostructures (B-1) was subjected to dispersing treatment at 100 MPa. The dispersed liquid was then added dropwise to 16 kg of water (20° C.) for solidification to afford a black solid. The black solid was dried under reduced pressure at 80° C. for 12 hours to afford a mixture of the fluorinated elastomer (A-1) and the fibrous carbon nanostructures (B-1).

### [Kneading]

Subsequently, 200.2 g of the above mixture, 20.0 g of a carbon black (D-1), 6.0 g of (E-2) as a cross-linking aid, 6.0 g of (E-1) as a co-cross-linking agent, and 2.0 g of an organic peroxide (C-1) were kneaded using a 15° C. open roll to afford a composition for forming a crosslinked material for an endoscope according to Example 1.

### [Preparation of Composition for Forming Crosslinked Material for Endoscope According to Examples 2 to 16 and Comparative Examples 1 to 6]

Compositions for forming a crosslinked material for an endoscope according to Examples 2 to 16 and Comparative Examples 1 to 6 were prepared similarly to the composition for forming a crosslinked material for an endoscope according to Example 1 except that a composition ratio indicated in Tables 1-1 to 1-3 were employed instead of the composition ratio of the composition for forming a crosslinked material for an endoscope according to Example 1 in the preparation of the composition for forming a crosslinked material for an endoscope according to Example 1.

### <Preparation of Sheet-Like Sample>

The obtained composition for forming a crosslinked material for an endoscope was placed into a mold and cross-linked at a temperature of 170° C. and a pressure of 10 MPa for 20 minutes to afford a sheet (150 mm length, 150 mm width, 2 mm thick). Next, the obtained sheet was transferred to a gear type oven and subjected to secondary cross-linking at 230° C. for 2 hours to afford a sheet-like sample (a crosslinked material for an endoscope).

### <Test 1>

The tests below were each conducted. Table 1 indicates the tests and the measurement results.

### <Flexibility Test (Durometer Type A Hardness Measurement)>

In accordance with JIS K 6253-3:2012, the test piece was measured for durometer type A hardness at a temperature of 23° C. For this measurement, a test piece in which three pieces of sheet-like samples punched out in a shape of dumbbell No. 3 were stacked to have a thickness of 6 mm was used.

### <Tear Strength Test>

In accordance with JIS K 6252:2015, the test piece was measured for tear strength (N/mm) at 23° C. The prepared sheet-like sample was punched out in an unnicked angle shape to provide a test piece used in this measurement. The measurement results were evaluated according to the following evaluation criteria. A to C are qualified in this test.

### —Evaluation Criteria—

A: 45 N/mm or more
B: 35 N/mm or more, and less than 45 N/mm
C: 25 N/mm or more, and less than 35 N/mm
D: less than 25 N/mm

### <Bending Durability Test>

The test was conducted in accordance with JIS K 6260:2017.

The test piece (length 140 to 155 mm, width 25 mm, thickness 6.3 mm) was prepared by cross-linking the composition for forming a crosslinked material for an endoscope at temperature of 170° C. with a pressure of 10 MPa for 20 minutes. A notch of 2.0 mm was made to be parallel to the width direction in the center of the test piece. This notch penetrates the test piece.

The notched test piece was repeatedly bent with a chuck distance (distance between chucks) of 65 mm and a stroke of 20 mm at 25° C. using De Mattia flex Cracking tester (FT-1503 manufactured by Ueshima-seisakusho Co., Ltd.), the notch (crack) was observed every 1000 times, and the number of times when the crack reached both ends in the test piece width direction was evaluated under the evaluation criteria below.

A to C are qualified in this test.

### —Evaluation Criteria—

A: 50,000 times or more
B: 20,000 times or more, and less than 50,000 times
C: 5,000 times or more, and less than 20,000 times
D: less than 5,000 times

### <Sterilization Treatment Resistance Test>

The sheet-like sample was punched in a shape of dumbbell No. 3 to afford a test piece. This test piece was repeatedly subjected to (1) and (2) below in this order 100 times.

(1) The test piece was bent 100 times similarly to the above-described <Bending durability test>.
(2) The test piece bent 100 times was subjected to hydrogen peroxide plasma sterilization treatment at room temperature using ADVANCED course of STERRAD (registered trademark) NX (trademark, manufactured by ASP).

A tensile test was conducted to a test piece (1) before subjected to (1) and (2) described above and a test piece (II) repeatedly subjected to (1) and (2) described above in this order 100 times with a tension speed of 20 mm/min and a distance between chucks of 20 mm using Autograph AGS-X (trademark, manufactured by Shimadzu Corporation).

The percentage of a breaking strength of the test piece (II) relative to a breaking strength of the test piece (I) ([breaking strength (MPa)] of test piece (II)]/[breaking strength (MPa) of test piece (I)]×100) was determined as a breaking strength retention, and sterilization treatment resistance was evaluated according to the following evaluation criteria. A to C are qualified in this test.

—Evaluation Criteria—

A: The breaking strength retention is 95% or more.
B: The breaking strength retention is 90% or more, and less than 95%.
C: The breaking strength retention is 85% or more, and less than 90%.
D: The breaking strength retention is less than 85%.

### <Test 2>

Tests (1) to (3) were conducted using the composition for forming a crosslinked material for an endoscope according to Example 4. They will be described below with reference to FIG. 1.

### (1) Angle Rubber Aptitude Test

The composition for forming a crosslinked material for an endoscope was compression-molded at 170° C. to prepare a tube having a length of 150 mm, an inner diameter of 12 mm, and a wall thickness of 0.5 mm. This tube was mounted to an angle portion (2b of FIG. 1) of an endoscope having an outer diameter of 12.8 mm. The insertion section 2 was operated to be upward (arrow direction of FIG. 1) from the original state and returned to the original state. This operation was repeated 5,000 times. The similar operation was performed downward 5,000 times, leftward 5,000 times, and rightward 5,000 times. Subsequently, the tube was removed from the endoscope and visually observed, and the tube was not damaged.

### (2) Bend Preventing Rubber Aptitude Test

The composition for forming a crosslinked material for an endoscope was compression-molded at 170° C. to prepare a tube having a length of 85 mm, a tip diameter: 8 mm, and a rear end diameter: 30 mm. This tube has an inner diameter increased (inclined) from the tip toward the rear end. This tube was mounted to an outer periphery of an elongated tubular member of the endoscope (portion sandwiched between dashed lines of FIG. 1, outer diameter in the insertion section 2 side: 12.8 mm, outer diameter in the main body operating section 4 side: 35 mm) to cover the insertion section 2 and the main body operating section 4. The insertion section 2 was operated to be upward (arrow direction of FIG. 1) from the original state and returned to the original state. This operation was repeated 2,000 times. The similar operation was performed downward 2,000 times, leftward 2,000 times, and rightward 2,000 times. Subsequently, the tube was removed from the endoscope and visually observed, and the tube was not damaged.

### (3) O-Ring Aptitude Test

The composition for forming a crosslinked material for an endoscope was compression-molded at 170° C. to prepare an O-ring having an inner diameter of 3 mm and a wire diameter of 2 mm. This O-ring was mounted to the air/water supply button 3 (outer diameter 20 mm) and removed. This attaching and removal were repeatedly performed 2,000 times and the O-ring was visually observed, and the O-ring was not damaged.

TABLE 1-1

| | | | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|---|---|
| Composition | Fluorinated | Kind | (A-1) | (A-1) | (A-1) | (A-1) | (A-1) | (A-1) | (A-1) | (A-1) | (A-1) |
| of mixture | elastomer (A) | Content | 100 | 100 | 100 | 100 | 100 | 100 | 100 | 100 | 100 |
| | | [mass parts] | | | | | | | | | |
| | Fibrous carbon | Kind | (B-1) | (B-1) | (B-1) | (B-1) | (B-1) | (B-1) | (B-2) | (B-1) | (B-1) |
| | nanostructures (B) | Content | 0.1 | 0.3 | 0.5 | 1.0 | 1.5 | 1.6 | 1.0 | 1.0 | 1.0 |
| | | [mass part(s)] | | | | | | | | | |
| | Organic peroxide (C) | Kind | (C-1) | (C-1) | (C-1) | (C-1) | (C-1) | (C-1) | (C-1) | (C-1) | (C-1) |
| | | Content | 1.0 | 1.0 | 1.0 | 1.0 | 1.0 | 1.0 | 1.0 | 0.4 | 2.0 |
| | | [mass part(s)] | | | | | | | | | |
| | Carbon black (D) | Kind | (D-1) | (D-1) | (D-1) | (D-1) | (D-1) | (D-1) | (D-1) | (D-1) | (D-1) |
| | | Content | 10.0 | 10.0 | 10.0 | 10.0 | 10.0 | 10.0 | 10.0 | 10.0 | 10.0 |
| | | [mass parts] | | | | | | | | | |
| | Other additives (E) | Kind | (E-1) | (E-1) | (E-1) | (E-1) | (E-1) | (E-1) | (E-1) | (E-1) | (E-1) |
| | | Content | 3.0 | 3.0 | 3.0 | 3.0 | 3.0 | 3.0 | 3.0 | 3.0 | 3.0 |
| | | [mass parts] | | | | | | | | | |
| | | Kind | (E-2) | (E-2) | (E-2) | (E-2) | (E-2) | (E-2) | (E-2) | (E-2) | (E-2) |
| | | Content | 3.0 | 3.0 | 3.0 | 3.0 | 3.0 | 3.0 | 3.0 | 3.0 | 3.0 |
| | | [mass parts] | | | | | | | | | |

| | | | | | | | | | | |
|---|---|---|---|---|---|---|---|---|---|---|
| Evaluation | Flexibility (surface hardness) | 63 | 65 | 67 | 71 | 74 | 75 | 70 | 70 | 72 |
| | Tear strength | C | B | B | A | A | A | C | B | A |
| | Bending durability | C | B | A | A | A | B | A | A | B |
| | Sterilization resistance | B | A | A | A | A | A | C | A | A |

Remarks: ‘Ex.’ means Example according to this invention.

TABLE 1-2

| | | | Ex. 10 | Ex. 11 | Ex. 12 | Ex. 13 | Ex. 14 | Ex. 15 | Ex. 16 |
|---|---|---|---|---|---|---|---|---|---|
| Composition | Fluorinated | Kind | (A-1) | (A-1) | (A-1) | (A-2) | (A-2) | (A-3) | A-3) |
| of mixture | elastomer (A) | Content | 100 | 100 | 100 | 100 | 100 | 100 | 100 |
| | | [mass parts] | | | | | | | |
| | Fibrous carbon | Kind | (B-1) | (B-1) | (B-1) | (B-1) | (B-1) | (B-1) | (B-1) |
| | nanostructures (B) | Content | 1.0 | 1.0 | 1.0 | 1.0 | 1.0 | 0.5 | 1.0 |
| | | [mass part] | | | | | | | |
| | Organic peroxide (C) | Kind | (C-1) | (C-1) | (C-1) | (C-2) | (C-2) | (C-1) | (C-1) |
| | | Content | 1.0 | 1.0 | 1.0 | 1.0 | 1.0 | 1.0 | 1.0 |
| | | [mass part] | | | | | | | |
| | Carbon black (D) | Kind | — | (D-1) | (D-1) | (D-1) | — | (D-1) | (D-1) |
| | | Content | — | 5.0 | 15.0 | 10.0 | — | 10.0 | 10.0 |
| | | [mass parts] | | | | | | | |
| | Other additives (E) | Kind | (E-1) | (E-1) | (E-1) | (E-1) | (E-1) | (E-1) | (E-1) |
| | | Content | 3.0 | 3.0 | 3.0 | 5.0 | 5.0 | 3.0 | 3.0 |
| | | [mass parts] | | | | | | | |
| | | Kind | (E-2) | (E-2) | (E-2) | (E-3) | (E-3) | (E-2) | (E-2) |
| | | Content | 3.0 | 3.0 | 3.0 | 1.0 | 1.0 | 3.0 | 3.0 |
| | | [mass part(s)] | | | | | | | |

| | | | | | | | | |
|---|---|---|---|---|---|---|---|---|
| Evaluation | Flexibility (surface hardness) | 66 | 68 | 73 | 71 | 66 | 45 | 52 |
| | Tear strength | B | A | A | A | B | C | B |
| | Bending durability | C | A | B | A | A | A | A |
| | Sterilization resistance | C | A | A | A | B | A | A |

Remarks: ‘Ex.’ means Example according to this invention.

TABLE 1-3

| | | | CEx. 1 | CEx. 2 | CEx. 3 | CEx. 4 | CEx. 5 | CEx. 6 |
|---|---|---|---|---|---|---|---|---|
| Composition | Fluorinated | Kind | (A-1) | (A-1) | (A-1) | (A-1) | (A-1) | (P-1) |
| of mixture | elastomer (A) | Content | 100 | 100 | 100 | 100 | 100 | 100 |
| | | [mass parts] | | | | | | |
| | Fibrous carbon | Kind | — | (B-1) | (B-1) | (B-1) | (B-1) | (B-1) |
| | nanostructures (B) | Content | | 0.05 | 2.4 | 5.0 | 10.0 | 1.0 |
| | | [mass part(s)] | | | | | | |
| | Organic peroxide (C) | Kind | (C-1) | (C-1) | (C-1) | (C-1) | (C-1) | (C-1) |
| | | Content | 1.0 | 1.0 | 1.0 | 1.0 | 1.0 | 1.0 |
| | | [mass part] | | | | | | |
| | Carbon black (D) | Kind | (D-1) | (D-1) | (D-1) | (D-1) | (D-1) | (D-1) |
| | | Content | 10.0 | 10.0 | 10.0 | 10.0 | 10.0 | 10.0 |
| | | [mass parts] | | | | | | |
| | Other additives (E) | Kind | (E-1) | (E-1) | (E-1) | (E-1) | (E-1) ) | (E-1 |
| | | Content | 3.0 | 3.0 | 3.0 | 3.0 | 3.0 | 3.0 |
| | | [mass parts] | | | | | | |
| | | Kind | (E-2) | (E-2) | (E-2) | (E-2) | (E-2) | (E-2) |
| | | Content | 3.0 | 3.0 | 3.0 | 3.0 | 3.0 | 3.0 |
| | | [mass parts] | | | | | | |

| | | | | | | | |
|---|---|---|---|---|---|---|---|
| Evaluation | Flexibility (surface hardness) | 61 | 62 | 82 | 96 | 98 | 95 |
| | Tear strength | D | D | A | A | A | B |
| | Bending durability | D | C | C | D | D | D |
| | Sterilization resistance | D | B | D | C | C | D |

Remarks: ‘CEx.’ means Comparative Example.

### <Notes for Tables>

### [Fluorinated Elastomer (A)]

(A-1) Viton GBL-200S (manufactured by Chemours, trademark)
(A-2) AFLAS 100S (manufactured by AGC Inc., trademark)
(A-3) Dyneon SFM-40L (manufactured by 3M Japan Limited, trademark)
(P-1) Non-fluorinated polyester elastomer, PELPRENE S-2001 (manufactured by TOYOBO CO., LTD.) (in Table 1-3, P-1 is described in the line of fluorinated elastomer (A) for comparison.)

### [Fibrous Carbon Nanostructures (B)]

(B-1) The above-prepared fibrous carbon nanostructures (B-1)
(B-2) Single-walled carbon nanotubes (manufactured by Nanointegris, trademark “Hipco Purified”)

Average diameter (Av): 1.1 nm

Value (3σ) obtained by multiplying standard deviation (σ) by 3: 0.2 nm, 3σ/Av: 0.18

Average length: 500 nm

### [Organic Peroxide (C)]

(C-1) 2,5-dimethyl-2,5-di(t-butylperoxy)hexane (trademark “PERHEXA 25B40” manufactured by NOR Corporation)
(C-2) 1,3-bis(t-butylperoxyisopropyl)benzene (trademark “Vul Cup 40KE” manufactured by GEO Specialty Chemicals Inc.)

### [Carbon Black (D)]

(D-1) “Thermax N990” (Thermax is a registered trademark in Japan, other countries, or both) manufactured by Cancarb Limited

### [Other Additives (E)]

(E-1) Triallyl isocyanurate (manufactured by Nihon Kasei Co., Ltd., trademark “TAIC” (TAIC is a registered trademark in Japan, other countries, or both))
(E-2) Zinc oxide (manufactured by Inoue Calcium Corporation, trademark “META-Z L40”)
(E-3) Sodium stearate (manufactured by DAINICHI CHEMICAL INDUSTRY CO., LTD.)

“-” indicates that the corresponding component is not included.

As shown in Table 1, it is found that the crosslinked material for an endoscope of the present invention has a sufficient flexibility as a constituting member of an endoscope, is also excellent in the tear strength, is less likely to be broken regardless of repeated bending operation, and further, is excellent in sterilization resistance.

The present invention has been described together with embodiments thereof. However, we do not intend to limit our invention in any of the details of the description unless otherwise specified. We believe that the invention should be broadly construed without departing from the spirit and scope of the invention as defined by the appended claims.

### DESCRIPTION OF SYMBOLS

1 Electronic endoscope (endoscope)
2 Insertion section
2a Flexible tube
2b Angle portion
2c Tip portion
3 Air/water supply button
4 Main body operating section
5 Universal cord

1. A crosslinked material for an endoscope, comprising:
a fluorinated elastomer; and
fibrous carbon nanostructures including single-walled carbon nanotubes,
wherein the amount of the fibrous carbon nanostructures in the crosslinked material is 0.1 parts by mass or more and less than 2.0 parts by mass per 100 parts by mass of the fluorinated elastomer, and
wherein a durometer type A hardness at 23° C. measured in accordance with JIS K 6253-3:2012 is 75A or less.

2. The crosslinked material for an endoscope according to claim 1, wherein the fibrous carbon nanostructures exhibit a convex upward shape in a t-plot obtained from an adsorption isotherm.

3. The crosslinked material for an endoscope according to claim 2, wherein the t-plot has a bending point in a range of 0.2 nm s t s 1.5 nm.

4. The crosslinked material for an endoscope according to claim 2, wherein total specific surface area S1 and internal specific surface area S2 of the fibrous carbon nanostructures, obtained from the t-plot, satisfy the condition 0.05≤S2/S1≤0.30.

5. The crosslinked material for an endoscope according to claim 1, wherein the fibrous carbon nanostructures have an average diameter of 2 nm or more and 10 nm or less.

6. The crosslinked material for an endoscope according to claim 1, wherein the amount of the fibrous carbon nanostructures in the crosslinked material is 0.1 parts by mass or more and less than 1.6 parts by mass per 100 parts by mass of the fluorinated elastomer.

7. The crosslinked material for an endoscope according to claim 1, comprising a carbon black,
wherein the amount of the carbon black in the crosslinked material is 5 parts by mass or more and 15 parts by mass or less per 100 parts by mass of the fluorinated elastomer.

8. An endoscope, comprising the crosslinked material for an endoscope according to claim 1.

9. A composition for forming the crosslinked material for an endoscope according to claim 1, comprising:
a fluorinated elastomer;
fibrous carbon nanostructures including single-walled carbon nanotubes; and
an organic peroxide.
